(19) United States
(12) Patent Application Publication
(10) Pub. No.: US 2017/0021123 A1
(43) Pub. Date: Jan. 26, 2017

(54) Respiratory Mask

(71) Applicant: Hsiner Co., Ltd., Taichung City (TW)

(72) Inventor: Eric Chang, Taichung City (TW)

(21) Appl. No.: 14/988,818

(22) Filed: Jan. 6, 2016

(30) Foreign Application Priority Data
Jul. 24, 2015 (TW) 104124112

(51) Int. Cl. A61M 16/08; A61M 16/06

(52) U.S. Cl. CPC A61M 16/0816; A61M 16/0622; A61M 16/0683; A61M 16/0633

(57) ABSTRACT

A respiratory mask includes a support frame, a mask pad, and an air duct. The support frame includes a base portion, an insert portion defining an air channel, a positioning pin having a hooked end, and two wing portions respectively disposed at lateral sides of the base portion. The mask pad has a main body, a through hole, and a face pad mounted on the main body. The main body has an open end portion abutting separably against the base portion and defining an opening for extension of the insert portion thereinto, and is clamped separably between the wing portions. The positioning pin engages removably the through hole. The air duct is connected to the support frame and communicates with the air channel.

### CROSS-REFERENCE TO RELATED APPLICATION

This application claims priority of Taiwanese Application No. 104124112, filed on Jul. 24, 2015.

### FIELD

The disclosure relates to a respiratory mask, more particularly to a respiratory mask that can be easily assembled and disassembled.

### BACKGROUND

A patient of Obstructive Sleep Apnea (OSA) generally wears a respiratory mask connected to a Continuous Positive Airway Pressure (CPAP) device during sleep. A conventional respiratory mask includes a support frame, a mask pad, and an air duct connected to the CPAP device. The support frame supports the mask pad and is connected to the air duct, and the mask pad is used to contact a face of a user. As such, the mask pad needs to be cleaned and replaced frequently to prevent breeding of germs due to passage or accumulation of humid exhaled air.

### SUMMARY

Therefore, the object of the disclosure is to provide a respiratory mask that can be easily used, assembled, and disassembled.

Accordingly, a respiratory mask of the disclosure includes a support frame, a mask pad, and an air duct. The support frame includes a ring-shaped base portion, an insert portion, a positioning pin, and two wing portions. The insert portion extends rearwardly from an inner edge of the base portion, and defines an air channel. The positioning pin extends rearwardly from a top end of the base portion, and has a hooked end distal from the base portion. The wing portions are respectively disposed at lateral sides of the base portion. The mask pad has a main body, a through hole, and a face pad. The main body has an open end portion abutting separably against the base portion of the support frame and defining an opening for extension of the insert portion of the support frame thereinto, and is clamped separably between the wing portions of the support frame. The through hole is formed in the main body. The positioning pin of the support frame engages removably the through hole such that the main body is fastened releasably on the support frame by the positioning pin and the wing portions. The face pad is mounted on the main body for contacting a face of a user. The air duct is connected to the support frame and communicates with the air channel.

### BRIEF DESCRIPTION OF THE DRAWINGS

Other features and advantages of the disclosure will become apparent in the following detailed description of the embodiment with reference to the accompanying drawings, of which:

FIG. 1 is an assembled perspective view of an embodiment of a respiratory mask according to the disclosure;

FIG. 2 is an exploded perspective view of the embodiment;

FIG. 3 is a perspective view of a support frame of the embodiment;

FIG. 4 is a fragmentary partly sectional view of the embodiment;

FIG. 5 is a side view of the embodiment in use; and

FIG. 6 is a front view of the embodiment in use.

### DETAILED DESCRIPTION

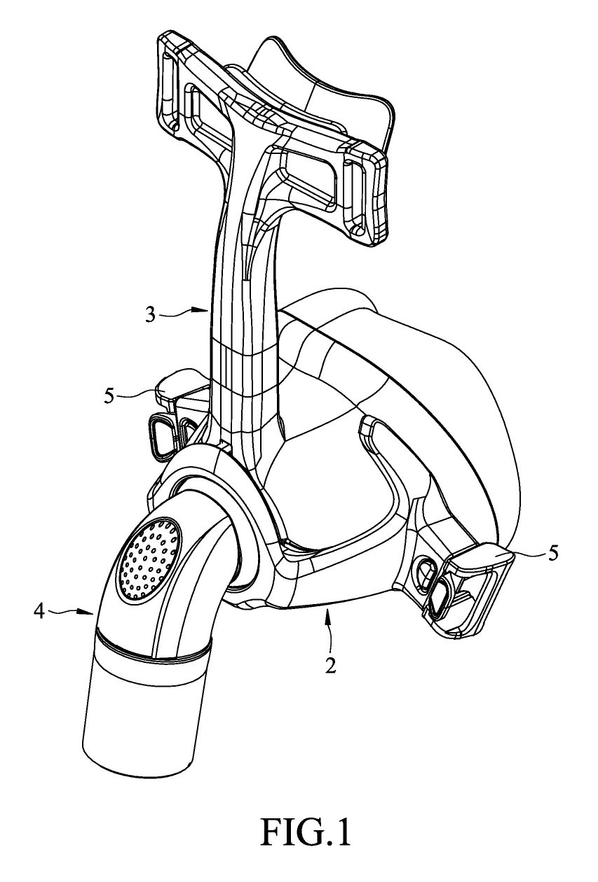

As shown in FIG. 1, an embodiment of a respiratory mask according to the present disclosure is adapted to cover a nasal area of a user's face and to be connected to a Continuous Positive Airway Pressure (CPAP) device (not shown), and is useful in treating a patient suffering from Obstructive Sleep Apnea (OSA). The CPAP device can apply a mild air pressure on a continuous basis to maintain an open airway for the patient who is unable to breathe spontaneously on his/her own.

The embodiment of the respiratory mask includes a support frame 2, a mask pad 3, an air duct 4, and a pair of strap-connecting members 5.

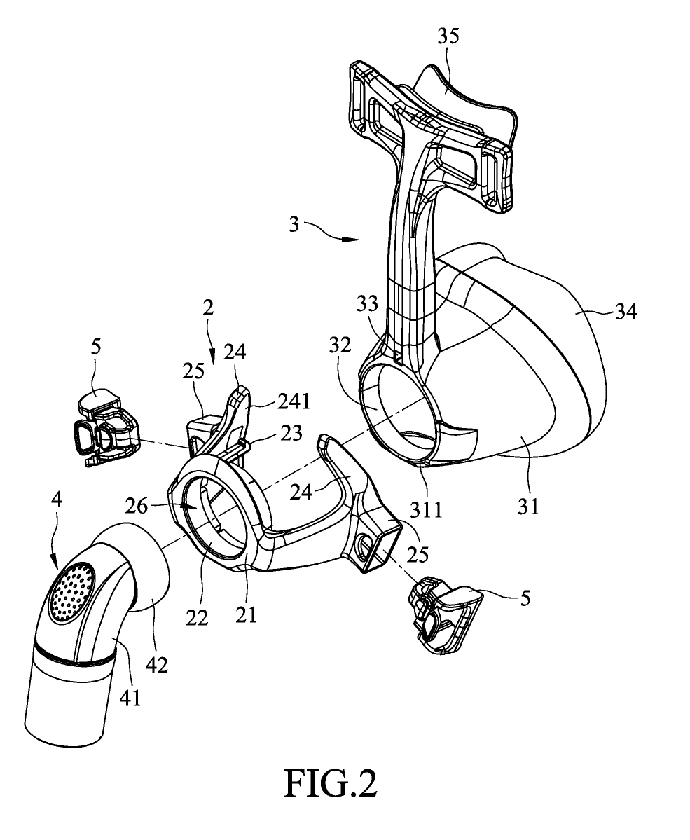

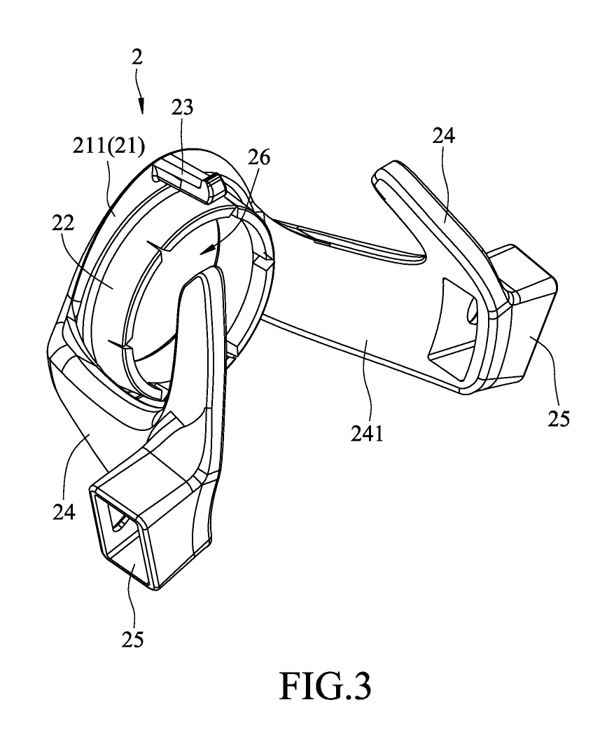

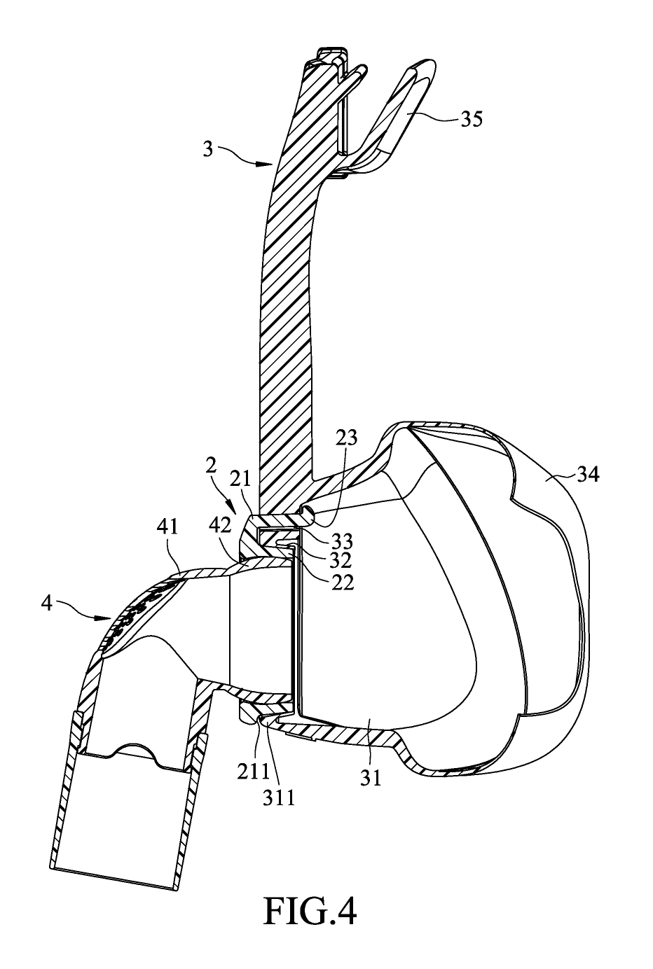

In this embodiment, referring to FIGS. 2 to 4, the support frame 2 is made of a lightweight, rigid plastic material, and includes a ring-shaped base portion 21 that has an abutment surface 211, an insert portion 22, a positioning pin 23, two wing portions 24, and two strap-connecting portions 25. The insert portion 22 extends rearwardly from an inner edge of the base portion 21, and defines an air channel 26. The positioning pin 23 extends rearwardly from a top end of the base portion 21, and has a hooked end distal from the base portion 21. The wing portions 24 of the support frame 2 are respectively disposed at lateral sides of the base portion 21. Each of the wing portions 24 extends rearwardly from a respective one of the lateral sides of the base portion 21, and has a bent end segment that is distal from the base portion 21 and bent upwardly, and a contact surface 241. As such, each of the wing portions 24 is substantially L-shaped, but the shape of the wing portions 24 may vary in other embodiments. The positioning pin 23 and the wing portions 24 are in a substantially triangular arrangement. The strap-connecting portions 25 are symmetrically and respectively formed at the bent end segments of the wing portions 24. Each of the strap-connecting portions 25 is configured as a groove.

The mask pad 3 has a main body 31, a sealing ring 32, a through hole 33, a face pad 34, and a forehead pad 35. The main body 31 has an open end portion 311 abutting separably against the abutment surface 211 of the base portion 21 of the support frame 2 and defining an opening for extension of the insert portion 22 of the support frame 2 thereinto, and is clamped separably between the wing portions 24 of the support frame 2. The sealing ring 32 extends rearwardly from the open end portion 311 of the main body 31 and is sleeved on the insert portion 22 of the support frame 2. The through hole 33 is formed in the main body 31, and the positioning pin 23 of the support frame 2 engages removably the through hole 33 such that the main body 31 is fastened releasably on the support frame 2 by the positioning pin 23 and the wing portions 24. The face pad 34 is mounted on the main body 31 for contacting the face of the user. The forehead pad 35 extends upwardly from the main body 31 and is adapted to abut against a forehead of the user. The mask pad 3 is made of elastic materials such as silica gel, with the main body 31 having a significant thickness, and the face pad 34 being thin so as to ease any discomfort. experienced by the user. It should be noted that, while the face pad 34 is exemplified to cover only the nasal area of the face of the user in this embodiment, the face pad 34 may cover both a nose and a mouth of the user in other embodiments. In the engagement between the positioning pin 23 of the support frame 2 and the through hole 33, the hooked end of the positioning pin 23 hooks onto an internal surface of the main body 31 to limit movements of the support frame 2. The contact surface 241 contacts separably the main body 31 of the mask pad 3. As such, by virtue of the abutment of the open end portion 311 against the abutment surface 211 of the base portion 21 of the support frame 2, the engagement between the positioning pin 23 and the through hole 33, the connection between sealing ring 32 and the insert portion 22, and the contact between the contact surface 241 and the main body 31 of the mask pad 3, the main body 31 can be firmly received in the support frame 2.

The air duct 4 is connected to the support frame 2, communicates with the air channel 26, and has a tube-connecting portion 41 and an annular lock portion 42. The tube-connecting portion 41 has an outer diameter that is smaller than an inner diameter of the base portion 21 defined by the inner edge of the base portion 21, a first end that is adapted to be connected to the CPAP device, and a second end that is opposite to the first end. The annular lock portion 42 extends from the second end of the tube-connecting portion 41, has an outer diameter that increases gradually in a direction away from the second end of the tube-connecting portion 41, and is slidably inserted into the insert portion 22 of the support frame 2 such that the user can easily adjust the position of the air duct 4 relative to the support. frame 2. It should be noted that, the air duct 4 may be fixed to the base portion 21 in other embodiments.

The strap-connecting members 5 are removably and respectively connected to the strap-connecting portions 25 of the support frame 2. In this embodiment, the strap-connecting members 5 are made of plastic and are removably and respectively fitted in the strap-connecting portions 25 of the support frame 2.

The structure of the support frame 2 is designed to prevent the air duct 4 from compressing the mask pad 3. When assembling the respiratory mask, the air duct 4 is firstly connected to the support frame 2, and the support frame 2 is then moved toward the mask pad 3 to engage the positioning pin 23 with the through hole 33 until the mask pad 3 abuts against the abutted surface 211 and the contact surface 241, thereby accomplishing the connection between the support frame 2 and the mask pad 3. When disassembling the respiratory mask, the only step required is to disengage the positioning pin 23 from the through hole 33 to separate the support frame 2 from the mask pad 3.

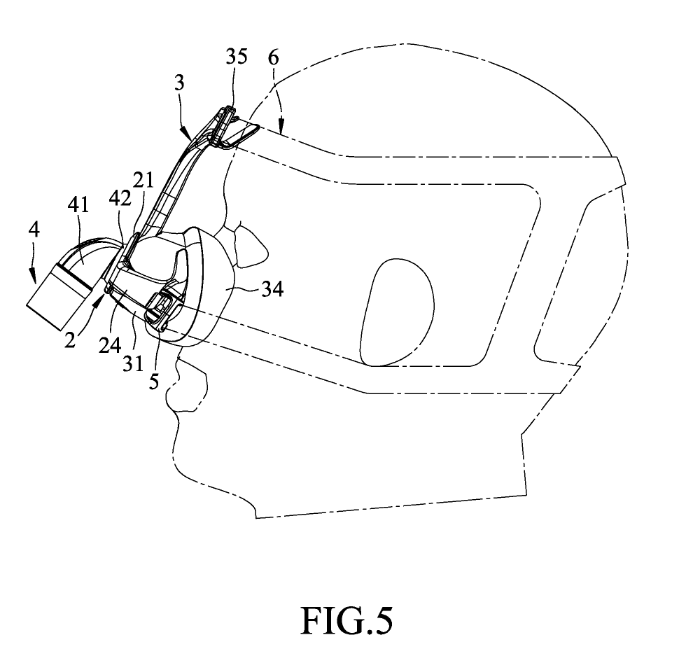

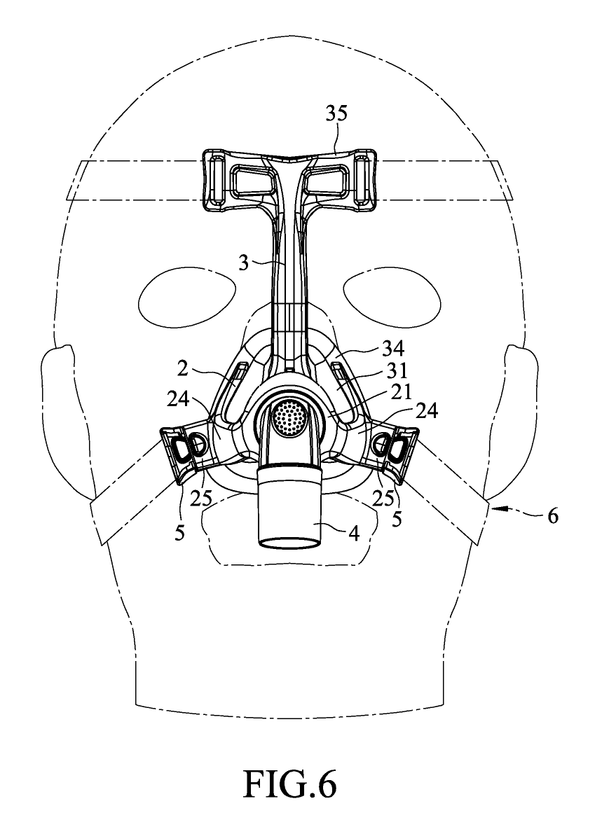

As shown in FIGS. 5 and 6, the respiratory mask of the disclosure can be worn on the face of the user using a strap 6. The strap 6 is separately coupled to the forehead pad 35 and the strap-connecting members 5. A length of the strap 6 is adjustable to fit users of different head sizes.

In conclusion, the respiratory mask can be conveniently assembled and disassembled, so that the respiratory mask can be easily worn, and the mask pad 3 can be quickly replaced.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A respiratory mask comprising:
a support frame including
a ring-shaped base portion,
an insert portion that extends rearwardly from an inner edge of said base portion, and that defines an air channel,
a positioning pin that extends rearwardly from a top end of said base portion, and that has a hooked end distal from said base portion, and
two wing portions that are respectively disposed at lateral sides of said base portion;
a mask pad having
a main body that has an open end portion abutting separably against said base portion of said support frame and defining an opening for extension of said insert portion of said support frame thereinto, and that is clamped separably between said wing portions of said support frame,
a through hole that is formed in said main body, said positioning pin of said support frame engaging removably said through hole such that said main body is fastened releasably on said support frame by said positioning pin and said wing portions, and
a face pad that is mounted on said main body for contacting a face of a user; and
an air duct connected to said support frame and communicating with said air channel.

23. The respiratory mask as claimed in claim 1, wherein:
each of said wing portions of said support frame extends rearwardly from a respective one of said lateral sides of said base portion, and has a bent end segment that is distal from said base portion and bent upwardly, and a contact surface that contacts separably said main body of said mask pad; and
said base portion has an abutment surface, said open end portion of said main body abutting separably against said abutment surface.

3. The respiratory mask as claimed in claim 1, wherein said mask pad further includes a sealing ring extending rearwardly from said open end portion of said main body and sleeved on said insert portion of said support frame.

4. The respiratory mask as claimed in claim 1, wherein said support frame further includes a pair or strap-connecting portions respectively formed at said wing portions.

5. The respiratory mask as claimed in claim 4, further comprising a pair of strap-connecting members removably and respectively connected to said strap-connecting portions of said support frame.

6. The respiratory mask as claimed in claim 1, wherein said mask pad further has a forehead pad extending upwardly from said main body and adapted to abut against a forehead of the user.

7. The respiratory mask as claimed in claim 1, wherein said air duct has:
a tube-connecting portion having an outer diameter that is smaller than an inner diameter of said base portion defined by said inner edge, said tube-connecting portion having a first end that is adapted to be connected to a Continuous Positive Airway Pressure device, and a second end that is opposite to said first end; and
an annular lock portion extending from said second end of said tube-connecting portion, having an outer diameter that increases gradually in a direction away from said second end of said tube-connecting portion, and being slidably inserted into said insert portion of said support frame.
